(19) United States
(12) Patent Application Publication
(10) Pub. No.: US 2024/0207478 A1
(43) Pub. Date: Jun. 27, 2024

(54) FRAGRANCE DIFFUSING DEVICE

(71) Applicant: Samwel Mwendwa, Marietta, GA (US)

(72) Inventor: Samwel Mwendwa, Marietta, GA (US)

(21) Appl. No.: 18/145,266

(22) Filed: Dec. 22, 2022

Related U.S. Application Data
(60) Provisional application No. 63/296,755, filed on Jan. 5, 2022.

(51) Int. Cl. A61L 9/12

(52) U.S. Cl. CPC A61L 9/12

(57) ABSTRACT

Disclosed embodiments may include a fragrance diffusing device including a fragrance reservoir and a vessel. The vessel may include a first substantially impermeable layer defining an internal space and comprising one or more openings; and a semi-permeable layer disposed within the internal space of the first substantially impermeable layer. The fragrance reservoir may be configured to be placed within the vessel with the semi-permeable layer disposed between the first substantially impermeable layer and the fragrance reservoir.

### CROSS-REFERENCE TO RELATED APPLICATION

This application claims priority under 35 U.S.C. § 119 to U.S. Patent Provisional Patent Application No. 63/296,755, filed Jan. 5, 2022, the entire contents of which is fully incorporated herein by reference.

### FIELD OF THE DISCLOSURE

The present disclosure relates generally to a long-lasting fragrance diffusing device.

### BACKGROUND

Typical air fresheners have very limited life before their fragrance is no longer detectable from a human nose. Typically, these last only a few short days requiring one to stock up on a large quantity of air fresheners and frequently replace them to maintain a base level of fragrance in a vehicle or other confined space. This causes unnecessary waste for landfills and inconvenience to the purchaser who has to replace their air freshener too frequently.

Accordingly, there is a need for a long-lasting fragrance diffusing device. Embodiments of the present disclosure are directed to this and other considerations.

### SUMMARY

Embodiments of the presently disclosed subject matter may provide a long-lasting fragrance diffusing device that uses multiple layers of permeable an impermeable material to slowly emit the fragrance from a fragrance source contained within. In some cases, the fragrance diffusing device may also serve as a decorative item in a vehicle.

Disclosed embodiments may include a fragrance diffusing device including a fragrance reservoir and a vessel. The vessel may include a first substantially impermeable layer defining an internal space and comprising one or more openings; and a semi-permeable layer disposed within the internal space of the first substantially impermeable layer. The fragrance reservoir may be configured to be placed within the vessel with the semi-permeable layer disposed between the first substantially impermeable layer and the fragrance reservoir.

### BRIEF DESCRIPTION OF THE DRAWINGS

The accompanying drawings, which are incorporated in and constitute a part of this specification, illustrate multiple embodiments of the presently disclosed subject matter and serve to explain the principles of the presently disclosed subject matter. The drawings are not intended to limit the scope of the presently disclosed subject matter in any manner.

FIG. 1 is a cut-away side view of a fragrance diffusing device according to an exemplary embodiment of the present disclosure.

FIG. 2 is a cut-away side view of a fragrance diffusing device according to an exemplary embodiment of the present disclosure.

### DETAILED DESCRIPTION

Although certain embodiments of the disclosure are explained in detail, it is to be understood that other embodiments are contemplated. Accordingly, it is not intended that the disclosure is limited in its scope to the details of construction and arrangement of components set forth in the following description or illustrated in the drawings. Other embodiments of the disclosure are capable of being practiced or carried out in various ways. Also, in describing the embodiments, specific terminology will be resorted to for the sake of clarity. It is intended that each term contemplates its broadest meaning as understood by those skilled in the art and includes all technical equivalents which operate in a similar manner to accomplish a similar purpose.

Herein, the use of terms such as “having.” “has,” “including,” or “includes” are open-ended and are intended to have the same meaning as terms such as “comprising” or “comprises” and not preclude the presence of other structure, material, or acts. Similarly, though the use of terms such as “can” or “may” are intended to be open-ended and to reflect that structure, material, or acts are not necessary, the failure to use such terms is not intended to reflect that structure, material, or acts are essential. To the extent that structure, material, or acts are presently considered to be essential, they are identified as such.

The components described hereinafter as making up various elements of the disclosure are intended to be illustrative and not restrictive. Many suitable components that would perform the same or similar functions as the components described herein are intended to be embraced within the scope of the disclosure. Such other components not described herein can include, but are not limited to, for example, similar components that are developed after development of the presently disclosed subject matter.

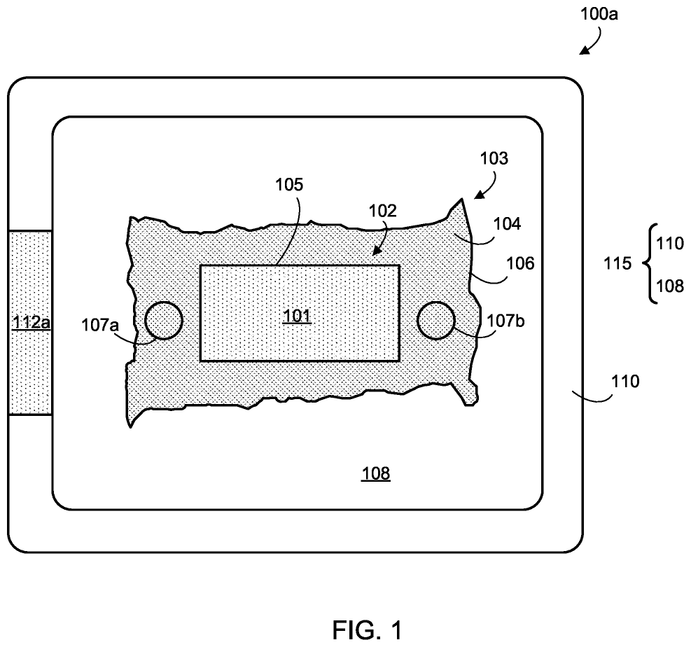

FIG. 1 is a cut away side view of a fragrance diffusing device 100a according to an exemplary embodiment of the present disclosure. The fragrance diffusing device 100a may include a fragrance reservoir 102 and a vessel 103.

The vessel 103 may include a first substantially impermeable layer 106 defining and surrounding an internal space show as the area inside of the first substantially impermeable layer 106. The first substantially impermeable layer 106 may include plastic (e.g., polymeric film), other impermeable material, or material with low permeability. The first substantially impermeable layer 106 also has one or more openings such as openings 107a, 107b. These openings may be of any shape or size. The vessel 103 may also include a semi-permeable layer 104 disposed within the internal space of the first substantially impermeable layer 106. The semi-permeable layer 104 may include cotton fibers, yarn (e.g., wool yarn), recycles plastic fibers, and/or other synthetic fibers. For example, the semi-permeable layer may include cotton balls. As another example, one or more layers of wool yarn can be used wound around the fragrance reservoir 102 as a semi-permeable layer 104 to control the release of the fragrance from the fragrance reservoir 102. Additionally, the fragrance reservoir 102 may be configured to be placed within the vessel with the semi-permeable layer 104 disposed between the first substantially impermeable layer 106 and the fragrance reservoir 102.

The fragrance reservoir 102 may include a permeable outer layer 105 and a fragrance emitting source 101 contained within or at least partially within the permeable outer layer 105.

The fragrance diffusing device 100a may also include a permeable container 115 configured to house the vessel 103 with the fragrance reservoir 102. The permeable container 115 may include an outer permeable portion 110 and an inner permeable portion 108. The inner permeable portion 108 may include foam (e.g., polymeric foam) and outer permeable portion 110 may include fabric made of cotton or other material. The inner permeable portion 108 may be positioned between the outer permeable portion 110 and the vessel 103. In some embodiments, permeable container 115, may include resealable opening 112a for placing the vessel 103 inside of the permeable container 115 or removing it from inside of the permeable container 115. The resealable opening 112a may utilize Velcro, buttons, zippers, or other suitable means to seal and unseal. The permeable container 115 may be decorative in nature. It may take the form of a plush animal, one or more dice, or the like. For example, fragrance diffusing device 100a may include a pair of hanging dice with a vessel 103 placed in one or both dice with a string connecting the pair of dice. The dice may be hung from a mirror of a vehicle from the string and from which a pleasant fragrance would emit slowly so that the vehicle is filled with a long-lasting aroma.

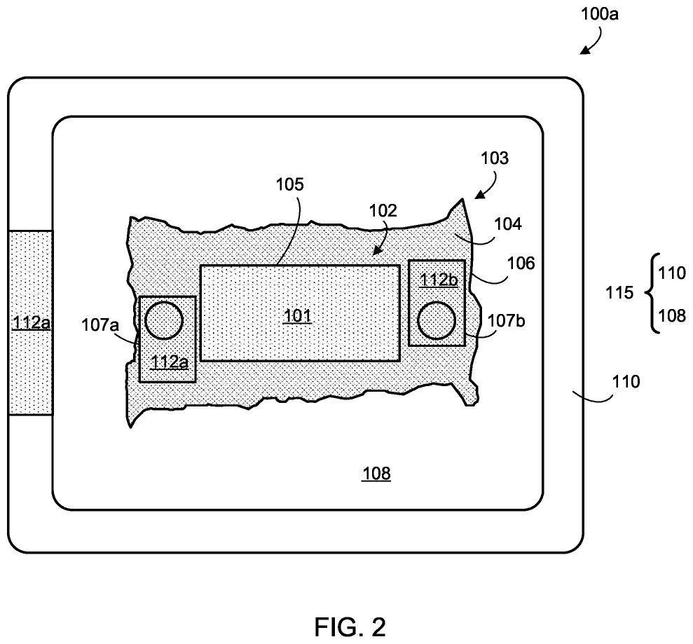

FIG. 2 is a cut away side view of a fragrance diffusing device 100b according to an exemplary embodiment. Fragrance diffusing device 100b is similar to fragrance diffusing device 100a shown in FIG. 1, except that fragrance diffusing device 100b include one or more sealing flaps 112a, 112b. Sealing flap 112a may include a resealing adhesive allowing the sealing flap to cover and seal opening 107a. Sealing flap 112b and opening 107b are exemplary and, if present, would work the same way. The one or more sealing flaps 112a, 112b may include a plastic or paper-based material (e.g., paper). In some embodiments, the one or more sealings flaps 112a, 112b may include a mechanical seal (e.g., air sealing interlocking male (stems) and female parts (grooves)).

What is claimed is:

1. A fragrance diffusing device comprising:
a fragrance reservoir; and
a vessel, wherein the vessel comprises:
a first substantially impermeable layer defining an internal space and comprising one or more openings; and
a semi-permeable layer disposed within the internal space of the first substantially impermeable layer;
wherein the fragrance reservoir is configured to be placed within the vessel with the semi-permeable layer disposed between the first substantially impermeable layer and the fragrance reservoir.

2. The fragrance diffusing device of claim 1, wherein the first substantially impermeable layer comprises plastic.

3. The fragrance diffusing device of claim 1, wherein the semi-permeable layer comprises cotton fibers, synthetic fibers, or both.

4. The fragrance diffusing device of claim 1, further comprising one or more second substantially impermeable layers overlapping the one or more openings.

5. The fragrance diffusing device of claim 1, further comprising an adhesive on at least a portion of the one or more second substantially impermeable layers.

6. The fragrance diffusing device of claim 1, wherein the first substantially impermeable layer comprises a perforated portion that extends partially from a first edge of the substantially impermeable layer to a second edge about a width of the first substantially impermeable layer.

7. The fragrance diffusing device of claim 1, further comprising a permeable container configured to house the vessel with the fragrance reservoir.

8. The fragrance diffusing device of claim 7, wherein the permeable container comprises a fabric portion and a foam portion between the fabric portion and the vessel.
